(19) United States
(12) Patent Application Publication
(10) Pub. No.: US 2024/0261136 A1
(43) Pub. Date: Aug. 8, 2024

(54) MOBILE PALMAR COOLING DEVICE

(71) Applicant: Arteria Technology, Inc., Boca Raton, FL (US)

(72) Inventors: Richard Lin, Houston, TX (US); Shriya Shah, Houston, TX (US); Spencer Shah, Houston, TX (US); Vivian T. W. Wong, Houston, TX (US); Sophia Zhou, Houston, TX (US)

(21) Appl. No.: 18/432,800

(22) Filed: Feb. 5, 2024

Related U.S. Application Data
(60) Provisional application No. 63/443,667, filed on Feb. 6, 2023.

(51) Int. Cl. A61F 7/10; A61F 7/00

(52) U.S. Cl. CPC A61F 7/10; A61F 2007/0036

(57) ABSTRACT

Described in this specification are mobile palmar cooling devices. An example device includes: a substantially cylindrical external container containing a liquid; an internal container disposed inside the external container, the internal container containing a phase change material; at least one submersible pump disposed inside the external container, the at least one submersible pump configured to circulate the liquid around the internal container; a power source connected to the at least one submersible pump, the power source configured to supply electrical power to the at least one submersible pump; and a controller connected to the at least one submersible pump, the controller configured to control the output of the at least one submersible pump.

### CROSS-REFERENCE TO RELATED APPLICATIONS

This application claims priority to and the benefit of U.S. Provisional Patent Applications No. 63/443,667, filed on Feb. 6, 2023, and entitled “MOBILE PALMAR COOLING TECHNOLOGY”, the entire contents of the which are incorporated herein by reference.

### FIELD

The present apparatus relates generally to portable cooling devices for controlling body core temperature in humans or animals.

### BACKGROUND

Elevated core body temperature can be induced by exercise, stress, or medical conditions that affect thermoregulation. When one's core body temperature heats up, pyruvate kinase—the protein needed to generate energy for one's muscles—becomes denatured, the brain activates fatigue signals, and heat exhaustion consequences result.

### SUMMARY

Currently, palmar cooling devices are not optimized and thus have not been widely adopted for use in performance enhancement or general health applications. From surveying users of existing palmar cooling devices, needs of consumers that are not currently addressed by the available technologies include the following: lower cost, increased mobility, greater ease of use, efficiency, and longer battery life. Existing technologies do not meet these requirements. The technologies described in this specification maintain efficiency and mobility, while addressing these needs, and thus provide an ideal low-cost solution. The described technologies provide a low-cost solution that bring palmar cooling to a larger population while retaining the crucial properties described above.

### BRIEF DESCRIPTION OF THE DRAWINGS

For clarity and understanding, some embodiments of the technologies are described in more detail in the appended drawings. These drawings should not, however, be considered as limiting the scope of the inventions and the inventions include any number of other equally useful embodiments.

FIG. 1 is a diagram of an example device for mobile palmar cooling including a PCM, pumps, and electronics, as described in this specification.

FIG. 2 is a photograph of a low-fidelity prototype of an example bottle-type device for mobile palmar cooling as described in this specification.

FIG. 3A is a diagram of an example device for mobile palmar cooling including a PCM and a rigid container, as described in this specification. FIG. 3B is a diagram of an example device for mobile palmar cooling including a PCM and a soft bag-type container, as described in this specification.

### DESCRIPTION

The concept of palmar cooling can be utilized to reduce core body temperature and combat heat exhaustion. In the palms of hands, there is a network of veins that specialize in heat transfer called arteriovenous anastomoses (AVAs). In response to increase in core body temperature, AVAs vasodilate and blood flows rapidly to the periphery of the body (e.g., the palms). Through circulation, blood cools and in turn cools one's core. To maximize this regulatory mechanism there are some criteria that must be met.

First, the temperature of cooling should be within 13-17° C. as vasoconstriction of the AVAs occurs below 12° C., which would prevent effective blood flow and cooling. Secondly, the palm should be in a relaxed state, as opposed to a fisted or fully extended shape, or mechanical constriction occurs. Finally, cooling should occur within 10 minutes as palmar cooling equates to ambient cooling beyond that time period. Previous studies have shown exciting results of palmar cooling enhancing performance and endurance in athletes. However, all existing palmar cooling technology is bulky, inefficient, or expensive, limiting the technology's environment and user demographic. Thus, there is a need to create an affordable and mobile palmar cooling device that rapidly and optimally lowers core body temperature in individuals experiencing elevated body temperature.

When core body temperature rises, heat exhaustion slowly sets in, making exercise increasingly difficult. To combat this condition, palmar cooling can be applied to rapidly and optimally lower core body temperature in individuals experiencing elevated body temperature.

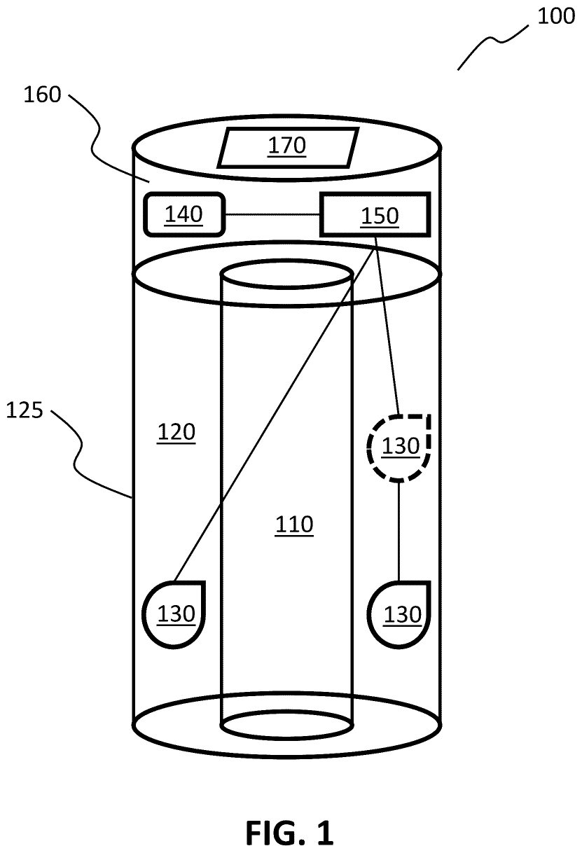

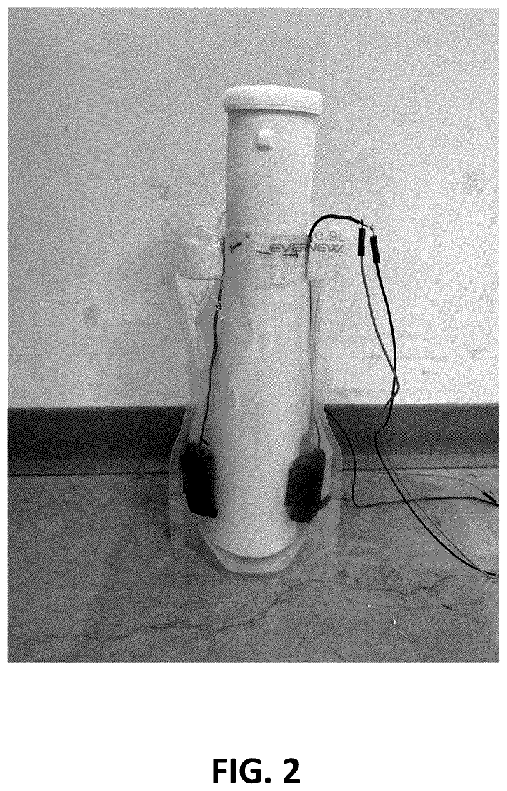

FIG. 1 is a diagram of an example device 100 for mobile palmar cooling as described in this specification. FIG. 2 is a photograph of a low-fidelity prototype of an example bottle-type device for mobile palmar cooling incorporating features of device 100, as described in this specification. In an example device, e.g., device 100, phase change material (PCM) is contained within an internal container, e.g., a cylinder 110. The PCM container is immersed in or surrounded by fluid, e.g., a liquid, in an external container, e.g., a cylindrical container 120 or bottle. In some implementations, the liquid is water or an aqueous solution. In some implementations, the liquid is or includes a gel, a paste, an emulsion, a suspension, an oil, and/or a semisolid. In some implementation, the liquid is thermochromic such that the liquid changes color according to the liquid temperature, e.g., to indicate when the device it “too warm” or otherwise losing effectiveness. The container 120 can be made from a glass, a metal, or a composite material. The container 120 can be made at least in part from a transparent, translucent, or opaque material. In some implementation, the container 120 is made at least in part for a thermochromic material such that the container surface 125 changes color according to the container temperature, e.g., to indicate when the device it “too warm” or otherwise losing effectiveness. One or more pumps, e.g., two pumps 130, disposed on or in the cylinder circulate the fluid to allow heat to be extracted from a user's palms and removed when the user places their hand on the cylinder surface 125.

Example devices as described herein can be portable, e.g., taking the form of a water bottle. This bottle can be large enough to be held by the hand and includes a flowing mechanism to flow the liquid, e.g., water, within the bottle that provides a way to extract heat away from the hand. Inside the bottle is a core (e.g., a cylinder) filled with phase change material (PCM). By coupling the heat of fusion associated with a phase change, a PCM can be used to absorb heat from the fluid (e.g., water), which will increase in temperature as heat from the hand is transferred to the fluid (e.g., water). The PCM/core (e.g., cylinder 110) can be kept in a freezer and can maintain its temperature for, e.g., 30 minutes of continuous use. To supply movement to the fluid that circulates around the PCM core inside the device (e.g., a bottle), one or more submersible water pumps 130 may be placed at various locations, e.g., heights, in the device/bottle, e.g., pumps that are capable of flow at 1-3 L/min. These example battery-powered pumps can have a voltage requirement of 3V and can be approximately 3 cm in width. In some implementations, some or all electrical components (e.g., a battery 140 and/or a controller 150 to control the one or more pumps) can be housed in the lid 160 of the bottle, in a watertight compartment. Relevant indicators, such as battery life and cooling completion, can displayed, e.g., on an LCD screen 170 on top of the lid. The inside of the lid can house necessary electrical components, such as the battery 140 and/or a PCB board containing the controller 150.

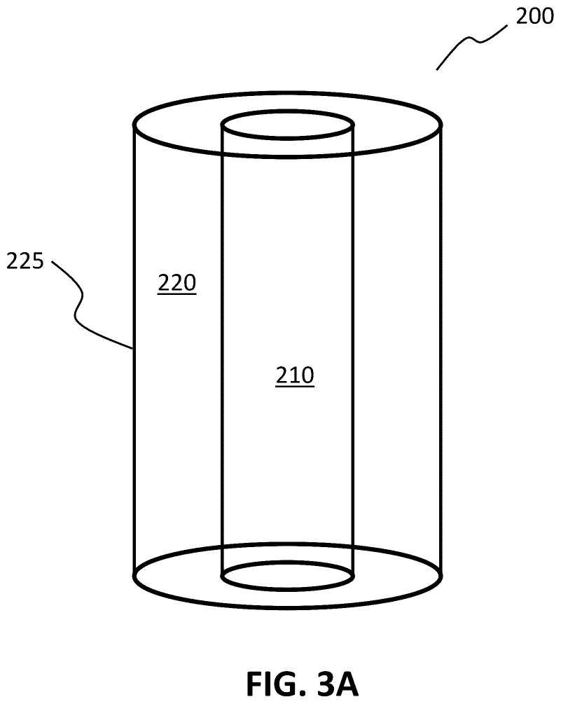

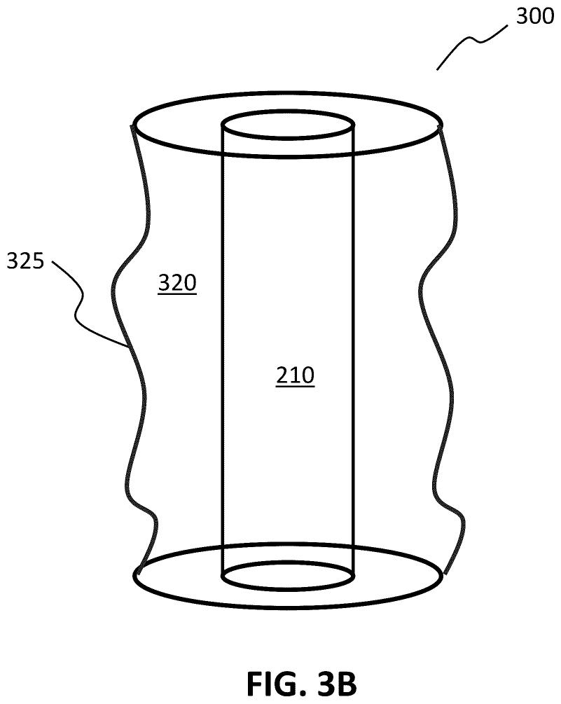

FIG. 3A-3B are diagrams of an example devices 200 and 300 for mobile palmar cooling as described in this specification. An example device 200 can include a PCM/heat sink 210 and an outer cylinder 220. An example device 300 can include a PCM/heat sink 210 and an outer soft bag 320. In some implementations, a device as described in this specification can include a combination of cylinder 220 and bag 320. The surface 225 of cylinder 220 on which the hand is placed can be rigid, while the surface 325 of bag 320 can be malleable or deformable by a human hand such that the surface conforms to the shape of a human hand when held. Because the outer cylinder/bag 320 is soft, when the user holds the bag, the movement of the fingers and/or palm can cause the liquid to move from the hand/surface 325 to the PCM 210 and induce convective cooling of the hand. The device 300 does not require a pump and electronics. In some implementations, the device 300 can include a pump, a power source, and electronics. The cylinder 220 and/or bag 320 can be made from a glass, a metal, or a composite material. The cylinder 220 and/or bag 320 can be made at least in part from a transparent, translucent, or opaque material. In some implementation, the cylinder 220 and/or bag 320 are made at least in part for a thermochromic material such that the container surface 125 changes color according to cylinder/bag temperature, e.g., to indicate when the device it “too warm” or otherwise losing effectiveness.

In an example implementation of a palmar cooling device as described in this specification, testing has been performed to evaluate the accuracy and cooling ability of an example PCM. From experiments, it has been shown that the PCM can reach an average of 14° C. (which falls within the optimal temperature range in a number of applications) for 4 of 5 trials at room temperature. Further, in an example implementation, the PCM alone can reach said example optimal cooling range temperature (13-17° C.) within an average of 5.73 minutes allowing for the user to be able to cool their palm relatively quickly after removing the PCM from the freezer. Testing was conducted on the capability of the prototype device to reduce the core body temperature of a user following exercise. Five subjects underwent a standardized treadmill run after which their body temperatures were measured (initial temperature after exercise). Then, each subject used an example palmar cooling device as described in this specification until their core body temperature had dropped by 1.5° C. The average time in which this 1.5° C. cooling was achieved was 10.44 minutes.

In an example implementation of a palmar cooling device as described in this specification, a surface of an example device as described in this specification, e.g., the surface 125, upon which the user places their palm, can maintain a temperature between 13-17° C. and can lower the user's elevated core body temperature by 2° C. in 5 minutes or less. Maintaining the temperature range can be accomplished by varying the combination of PCM melting temperature and/or the device surface material/thickness to meet this objective. Moreover, a device as described in this specification can have different shapes and/or dimensions to be compatible with different users having palms of varying sizes.

A device as described in this specification can include user-friendly features to address ease of use in the form of an insulating cover on the device surface and/or the electronic interface. The insulating cover can surround the device to keep it cold, with an adjustable zip-up opening for the user to insert their hand onto the palmar interface. A display screen can be added that shows cooling progress so that the user knows when they can remove their hand.

As a result, the technologies described in this specification have the potential to benefit, e.g., recreational athletes both in the gym and on the field by reducing their recovery, thereby increasing their exercise capacity.

1. A mobile palmar cooling device comprising:
a substantially cylindrical external container containing a liquid; and
an internal container disposed inside the external container, the internal container containing a phase change material.

2. The device of claim 1, wherein the external container is rigid.

3. The device of claim 1, wherein the external container comprises a bag configured to be deformable by an unassisted human hand.

4. The device of claim 1, wherein the external container comprises a thermochromic material.

5. The device of claim 1, comprising:
at least one submersible pump disposed inside the external container, the at least one submersible pump configured to circulate the liquid around the internal container;
a power source connected to the at least one submersible pump, the power source configured to supply electrical power to the at least one submersible pump; and
a controller connected to the at least one submersible pump, the controller configured to control the output of the at least one submersible pump.

6. The device of claim 5, wherein the one or more submersible pumps are disposed on an inner surface of the external container.

7. The device of claim 1, wherein the liquid is water.

8. The device of claim 1, wherein the liquid is a thermochromic liquid.

9. A mobile palmar cooling device comprising:
an external container configured to contain a liquid;
an internal container disposed inside the external container, the internal container configured to contain a phase change material;
at least one submersible pump disposed inside the external container, the at least one submersible pump configured to circulate the liquid around the internal container;
a power source connected to the at least one submersible pump, the power source configured to supply electrical power to the at least one submersible pump; and
a controller connected to the at least one submersible pump, the controller configured to control the output of the at least one submersible pump.
